(19) United States
(12) Patent Application Publication
(10) Pub. No.: US 2019/0240328 A1
(43) Pub. Date: Aug. 8, 2019

(54) NOVEL HUMANIZED ANTI-EBOLA ANTIBODIES USEFUL IN PREVENTING EBOLA INFECTIONS

(71) Applicant: The Trustees of the University of Pennsylvania, Philadelphia, PA (US)

(72) Inventors: Maria P. Limberis, Philadelphia, PA (US); Anna P. Tretiakova, Woburn, MA (US)

(21) Appl. No.: 16/335,767

(22) PCT Filed: Sep. 22, 2017
(86) PCT No.: PCT/US17/52991

§ 371 Date: Mar. 22, 2019

Related U.S. Application Data
(60) Provisional application No. 62/399,362, filed on Sep. 24, 2016.

(51) Int. Cl. A61K 39/42; C12N 15/85; C07K 16/10; A61P 31/14

(52) U.S. Cl. CPC A61K 39/42; C12N 15/85; A61K 2039/507; A61P 31/14; C07K 16/1081

(57) ABSTRACT

A recombinant vector encoding a humanized 2G4 anti-ebola antibody (H2G4) is provided. Also provided is a recombinant vector encoding a humanized 4G7 anti-ebola antibody (H4G7). Compositions containing at least one of these humanized anti-ebola antibodies are provided. Also described are methods of improving survival against ebola infection in a human population which use these antibodies and compositions.

### STATEMENT OF FEDERALLY FUNDED RESEARCH

This work was supported by a research grant from DARPA/DOD #64047-LS-DRP.01. The US government may have certain rights in this invention.

### BACKGROUND OF THE INVENTION

An effective strategy for preventing or controlling viral outbreaks is the availability of a vaccine that elicits a broadly neutralizing antibody response against the viral pathogen. The confluence of technological advances in anti-viral antibody isolation and gene transfer vector delivery creates a novel platform for a rapid response to emerging virus pandemics and new biothreats. The use of adeno-associated virus (AAV) vectors to deliver antiviral antibodies (Abs) to confer prophylaxis against infectious diseases including influenza and HIV has been described [Balazs, A. B., et al, Nat Med 2014, 20 (3), 296-300; Balazs, A. B., et al, Nature 2012, 481 (7379), 81-84; Balazs, et al, Nat Biotechnol 2013, 31 (7), 647-652; Limberis, M. P., et al., Sci Transl Med 2013, 5 (187), 187ra172; Adam, V. S., et al. Clin Vaccine Immunol 2014, 21 (11), 1528-1533; Limberis, M. P., et al. Clin Vaccine Immunol 2013, 20 (12), 1836-1837; Horwitz, J. A., et al. Proc Natl Acad Sci USA 2013, 110 (41), 16538-16543].

Anti-ebola murine monoclonal antibodies (mAbs) which recognize the surface glycoprotein of the Zaire EBOV (ZEBOV) have been studied for treatment of EBOV infections. Three such mAbs 4G7 [Qiu, X., et al. Sci Transl Med 2016, 8 (329), 329ra333; Qiu, X., et al. Nature 2014, 514 (7520), 47-53; Qiu, X., et al. Sci Transl Med 2013, 5 (207), 207ra143], 2G4 [Qiu, X., et al, Clin Immunol 2011, 141 (2), 218-227] and c13C6 [Olinger, G. G., Jr., et al. Proc Natl Acad Sci USA 2012, 109 (44), 18030-18035] have been described as being different from one another, although each reportedly recognizes the ZEBOV surface glycoprotein. See, also, U.S. Pat. Nos. 8,513,391; 9,249,214, 9,145,454. These antibodies have been described as providing therapeutic effects in EBOV challenge mouse and macaque studies.

The use of a murine antibody in humans raises concerns regarding an immune response being generated to the antibody, thereby reducing the effectiveness of the murine antibody. Thus, humanization of murine anti-ebola antibodies has been proposed. See, e.g., G. Chen et al, ACS Chem Biol., 2014 Oct. 17; 9 (10): 2263-2273.

However, concerns have been raised about the effectiveness of humanized antibodies. See, e.g., D R Getts, et al, MABs 2010 November-December; 2 (6): 682-694.

U.S. Pat. No. 8,513,397 describes the use of tobacco plants for production of antibodies against ebola. See, e.g., U.S. Pat. No. 8,513,397.

What are needed are effective methods increasing survival rates in human populations during ebola outbreaks and/or methods for preventing ebola infection.

### SUMMARY OF THE INVENTION

In one aspect, the invention provides a recombinant vector which comprises an expression cassette comprising the nucleic acid sequence encoding a humanized anti-ebola antibody under the control of regulatory sequences which direct expression of the antibody in target cells, wherein the anti-ebola antibody is selected from:
(a) a humanized 2G4 anti-ebola antibody (H2G4) comprising:
   (i) a heavy chain comprising a variable region having the amino acid sequence of SEQ ID NO: 6 (H2G4VH); and
   (ii) a light chain comprising a variable region having the amino acid sequence of SEQ ID NO: 8 (H2G4VL); or
(b) a humanized 4G7 anti-ebola antibody (H4G7) comprising:
   (i) a heavy chain comprising a variable region having the amino acid sequence of SEQ ID NO: 2 (H4G7VH); and
   (ii) a light chain comprising a variable region having the amino acid sequence of SEQ ID NO: 4 (H4G7VL).

In another aspect, a composition is provided which comprises a carrier, diluent, excipient and/or preservative and the recombinant vector. In certain embodiments, the composition comprises more than one anti-ebola component.

In a further aspect, a method is provided for preventing ebola infection comprising delivering an effective amount of the recombinant vector described herein to a subject at risk of infection.

In yet another aspect, a method is provided for improving survival rates against ebola in a human population comprising delivering an effective amount of the recombinant vector. In one embodiment, the method involves administering the prior to infection with ebola.

In still another aspect, a recombinant humanized antibody is provided which is useful in preventing infection with ebola virus. The antibody is selected from:
(a) a humanized 2G4 anti-ebola antibody (H2G4) comprising:
   (i) a heavy chain comprising a variable region having the amino acid sequence of SEQ ID NO: 6 (2G4VH); and
   (ii) a light chain comprising a variable region having the amino acid sequence of SEQ ID NO: 8 (2G4VL); or
(b) a humanized 4G7 anti-ebola antibody (H4G7) comprising:
   (i) a heavy chain comprising a variable region having the amino acid sequence of SEQ ID NO: 2 (4G7VH); and
   (ii) a light chain comprising a variable region having the amino acid sequence of SEQ ID NO: 4 (4G7VL).

In a further aspect, a composition comprises an excipient, carrier, diluent, and/or preservative and a recombinant antibody as descried herein.

In still a further aspect, a method is provided for preventing ebola infection comprising delivering an effective amount of an anti-ebola antibody as provided herein to a subject at risk of infection.

In a further aspect, a method is provided for improving survival rates against ebola in a human population comprising delivering an effective amount of an anti-ebola antibody as described herein. In one embodiment, anti-ebola antibody is delivered prior to infection with ebola.

Still other aspects and advantages of the invention will be apparent from the following detailed disclosure of the invention.

### BRIEF DESCRIPTION OF THE DRAWINGS

FIGS. 1A to 1F illustrate AAV9-mediated prophylaxis against challenge with mouse adapted (MA)-ebola virus Zaire (ZEBOV). FIG. 1A provides schematic of the AAV-Ab structure. FIG. 1B provides timeline of the AAV9-mediated prophylaxis and MA-ZEBOV challenge in mice. FIG. 1C provides body weight changes of MA-ZEBOV-challenged mice with or without protection of intramuscular administration of anti-ebola vector. BALB/c mice (n=6/group) were injected intramuscularly (IM) with various combinations of AAV9 vector (10¹¹ GC each) expressing either 2G4, 4G7 or c13C6 antibodies (Ab). Two weeks later, mice (n=6/group) were challenged intraperitoneally (IP) with 1,000 LD50 of MA-ZEBOV. Positive control mice (n=6) were injected with 100 μg of ZMapp two days after the MA-ZEBOV challenge. FIG. 1D provides survival curve of MA-ZEBOV-challenged mice with or without protection of intramuscular administration of anti-ebola vector. Experiment was performed as described in FIG. 1C. Naïve mice (n=6) died within 4 days of the challenge. The various combinations of AAV vectors expressing the 2G4, 4G7 and c13C6 Abs, similarly to ZMapp, prolonged time to death to 17 days. FIG. 1E provides body weight changes of MA-ZEBOV-challenged mice with or without protection of intranasal administration of anti-ebola vector. BALB/c mice (n=6/group) were injected intranasally (IN) with various combinations of AAV9 (10¹¹ GC each) vector expressing 2G4, 4G7 or c13C6. Two weeks later, mice were challenged intranasally with 1,000 LD50 of MA-ZEBOV. Positive control mice (n=6) were injected with 100 μg of ZMapp two days after the MA-ZEBOV challenge. FIG. 1F provides survival curve of MA-ZEBOV-challenged mice with or without protection of intranasal administration of anti-ebola vector. Experiment was performed as described in FIG. 1E. The combination of AAV9 vectors expressing 4G7 and c13C6 conferred full survival against the MA-ZEBOV challenge but was accompanied by significant weight loss (22%). Administration of mice (n=6/group) with either AAV9 vectors expressing the 2G4, 4G7 and c13C6 Abs or AAV9 vectors expressing the 2G4 and c13C6 Abs resulted in 83% survival. Notably the weight loss presented by the mice administered these two AAV9 regimens was similar to that exhibited by the mice treated ZMapp. IN: intranasal, IP: intraperitoneally, IM: intramuscularly. VH: variable heavy chain, VL: variable light chain, CH1, 2 or 3: constant heavy chain 1, 2 or 3, CL: constant light chain, F2A: Foot-and-mouth disease virus 2A (F2A), pA: poly A signal, ITR: inverted terminal repeat.

FIGS. 2A to 2F illustrate humanization of 2G4 to improve AAV9-mediated prophylaxis against challenge with MA-ZEBOV. BALB/c mice (n=8/group) were injected IN (FIG. 2A) or IM (FIG. 2B) with 3×10¹⁰ GC of either AAV9.2G4 (triangles) or AAV9.h2G4 (circles; h2G4, humanized 2G4) to evaluate the kinetics and levels of expression in serum up to day 28. FIG. 2C provides body weight changes of MA-ZEBOV-challenged mice with or without protection of intramuscular administration of AAV9.2G4 and AAV9.c13C6 or AAV9.h2G4 and AAV9.c13C6. BALB/c Rag mice (n=8/group) were injected IM with 10¹¹ GC each of either AAV9.2G4 and AAV9.c13C6 or AAV9.h2G4 and AAV9.c13C6. Two weeks later, mice were challenged IP with 1,000 LD50 of MA-ZEBOV. Positive control mice (n=8) were injected with 100 μg of ZMapp two days after the MA-ZEBOV challenge. FIG. 2D provides survival curve of MA-ZEBOV-challenged mice with or without protection of intramuscular administration of AAV9.2G4 and AAV9.c13C6 or AAV9.h2G4 and AAV9.c13C6. Experiment was performed as described in FIG. 2C. Administration of AAV9.h2G4 and AAV9.c13C6 resulted in 100% survival with no significant weight loss. ZMapp did not protect the mice (n=8) against the challenge as mice were provided only therapeutic dose. FIG. 2E provides body weight changes of MA-ZEBOV-challenged mice with or without protection of intranasal administration of AAV9.2G4 and AAV9.c13C6 or AAV9.h2G4 and AAV9.c13C6.BALB/c mice (n=8/group) were injected IN with 10¹¹ GC each of either AAV9.2G4 and AAV9.c13C6 or AAV9.h2G4 and AAV9.c13C6. Two weeks later, mice were challenged IN with 1,000 LD50 of MA-ZEBOV. Positive control mice (n=8) were injected with 100 μg of ZMapp two days after the MA-ZEBOV challenge. FIG. 2F provides survival curve of MA-ZEBOV-challenged mice with or without protection of intranasal administration of AAV9.2G4 and AAV9.c13C6 or AAV9.h2G4 and AAV9.c13C6. Experiment was performed as described in FIG. 2E. 10% of naïve mice (n=8/group) survived the challenge and both the AAV9-prophylaxis regimens and ZMapp resulted in 87.5% survival. Interestingly, mice given AAV9.h2G4 and AAV9.c13C6 (n=8) presented with minimum weight loss, followed by the regimen of AAV9.2G4 and AAV9.c13C6 (n=8) which resulted in a 7.5% weight loss. Mice treated with a single dose of ZMapp (n=8) presented with an 18% weight loss. IN: intranasal, IP: intraperitoneally, IM: intramuscularly.

FIGS. 3A-3B provide a series of alignments. FIG. 3A shows the sequences of 2G4VH (SEQ ID NO: 6) or 2G4VL (SEQ ID NO: 8), a murine Germ line (SEQ ID NO: 17 for VH and SEQ ID NO: 19 for VL), human Germ line (SEQ ID NO: 18 for VH and SEQ ID NO: 20 for VL), and final sequences (SEQ ID NO: 25 for VH and SEQ ID NO: 26 for VL). FIG. 3BB shows the sequences of 4G7VH (SEQ ID NO: 21 for VH and SEQ ID NO: 23 for VL) or 4G7VL (SEQ ID NO: 22 for VH and SEQ ID NO: 24 for VL), a murine Germ line, human Germ line, and final sequences (SEQ ID NO: 27 for VH and SEQ ID NO: 28 for VL).

### DETAILED DESCRIPTION OF THE INVENTION

Novel anti-ebola antibodies useful in treating ebola infection, preventing infection with ebola and/or improving survival rates in at-risk populations is provided herein. In certain embodiments, a humanized antibody is provided which has the advantage of preserving the effectiveness or activity (e.g., being bioequivalent) to the murine antibody from which it is derived while reducing the disadvantages typically associated with non-human antibodies when delivered to human patients, including, e.g., one or more of reduced effectiveness, induction of immune response to the antibody, and the like.

In one embodiment, a recombinant humanized antibody is provided which is useful in treatment and/or prevention of ebola infection. In one embodiment, the recombinant humanized antibody is a humanized 2G4 anti-ebola antibody (H2G4) comprising: a heavy chain comprising, at a minimum, a variable domain having the amino acid sequence of SEQ ID NO: 6 (2G4VH) and a light chain comprising, at a minimum, a variable domain having the amino acid sequence of SEQ ID NO: 8 (2G4VL). In certain embodiments, the antibody is a full-length antibody. In other embodiments, the antibody is an immunoadhesin. In still other embodiments, the antibody is a bispecific antibody. In certain embodiments, the heavy chain further comprises the constant domain of SEQ ID NO: 11. In certain embodiments, the light chain further comprises the constant domains of SEQ ID NO: 10.

Another suitable recombinant humanized 4G7 anti-ebola antibody (4G7) comprises a heavy chain comprising a variable domain having the amino acid sequence of SEQ ID NO: 2 (4G7VH); and a light chain comprising a variable domain having the amino acid sequence of SEQ ID NO: 4 (4G7VL). In certain embodiments, the antibody is a full-length antibody. In other embodiments, the antibody is an immunoadhesin. In still other embodiments, the antibody is a bispecific antibody. In certain embodiments, the heavy chain further comprises the constant domain of SEQ ID NO: 14. In certain embodiments, the light chain further comprises the constant domains of SEQ ID NO: 13.

Encompassed within the scope of the invention are the nucleic acid sequences encoding the 4G7 and 2G4 amino acid sequences described herein. These sequences may include DNA (e.g., cDNA) and RNA (e.g., mRNA) sequences. Such sequences may be used to express the immunoglobulins in vitro or for producing vectors which deliver and direct expression of the immunoglobulins in vivo.

An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

An “immunoadhesin” is a chimeric, antibody-like molecule that combines the functional domain of a binding protein, usually a receptor, ligand, scFv, variable heavy or light chains, or cell-adhesion molecule, with immunoglobulin constant domains, usually including the hinge and Fc regions.

A “fragment antigen-binding” (Fab) fragment” is a region on an antibody that binds to antigens. It is composed of one constant and one variable domain of each of the heavy and the light chain.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

As used herein, an “expression cassette” refers to a nucleic acid molecule which comprises an immunoglobulin gene(s) (e.g., an immunoglobulin variable region, an immunoglobulin constant region, a full-length light chain, a full-length heavy chain or another fragment of an immunoglobulin construct), promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the immunoglobulin sequences described herein flanked by packaging signals of the viral genome and other expression control sequences such as those described herein.

As described above, the term “about” when used to modify a numerical value means a variation of ±10%, unless otherwise specified.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

The humanized antibodies provided may be engineered into a suitable vector element for in vitro antibody production. Any suitable vector system and production cell culture, e.g., bacterial (e.g., E coli), mammalian (e.g., CHO), yeast, or insect cells, may be selected.

A vector as described herein can comprise one or more nucleic acid sequences, each of which encodes one or more of the heavy and/or light chain polypeptides, or other polypeptides, of an immunoglobulin construct. Suitably, a composition contains one or more vectors which contain all of the polypeptides which form an active immunoglobulin construct in vivo. For example, a full-length antibody consists of four polypeptides: two identical copies of a heavy (H) chain polypeptide and two copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. In this respect, an AAV vector as described herein can comprise a single nucleic acid sequence that encodes the two heavy chain polypeptides (e.g., constant variable) and the two light chain polypeptides of an immunoglobulin construct. Alternatively, the vector can comprise a first expression cassette that encodes at least one heavy chain constant polypeptides and at least one heavy chain variable polypeptide, and a second expression cassette that encodes both light chain polypeptides of an immunoglobulin construct. In yet another embodiment, the vector can comprise a first expression cassette encoding a first heavy chain polypeptide, a second expression cassette encoding a second heavy chain polypeptide, a third expression cassette encoding a first light chain polypeptide, and a fourth expression cassette encoding a second light chain polypeptide.

Typically, an expression cassette for an AAV vector comprises an AAV 5′ inverted terminal repeat (ITR), the immunoglobulin construct coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used.

Where a pseudotyped AAV is to be produced, the ITRs in the expression are selected from a source which differs from the AAV source of the capsid. For example, AAV2 ITRs may be selected for use with an AAV capsid having a particular efficiency for targeting CNS or tissues or cells within the CNS. In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The expression cassette typically contains a promoter sequence as part of the expression control sequences, e.g., located between the selected 5′ ITR sequence and the immunoglobulin construct coding sequence. Tissue specific promoters, constitutive promoters, regulatable promoters [see, e.g., WO 2011/126808 and WO 2013/04943], or a promoter responsive to physiologic cues may be used may be utilized in the vectors described herein. In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., CMV enhancer.

These control sequences are “operably linked” to the immunoglobulin construct gene sequences. As used herein, the term “operably linked” refers to both expression control sequences that are contiguous with the gene of interest and expression control sequences that act in trans or at a distance to control the gene of interest.

In one embodiment, a self-complementary AAV is provided. This viral vector may contain a Δ5′ ITR and an AAV 3′ ITR. In another embodiment, a single-stranded AAV viral vector is provided. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

A number of suitable purification methods may be selected. Examples of suitable purification methods are described, e.g., in U.S. Patent Applications No. 62/266,351 (AAV1); 62/266,341 (AAV8); 62/266,347 (AAVrh10); and 62/266,357 (AAV9), which are incorporated by reference herein.

In certain embodiments, an immunoglobulin-containing expression cassette contains at least one internal ribosome binding site, i.e., an IRES, located between the coding regions of the heavy and light chains. In other embodiments, the heavy and light chain may be separated by a furin-2a self-cleaving peptide linker [see, e.g., Radcliffe and Mitrophanous, Gene Therapy (2004), 11, 1673-1674]. The expression cassette may contain at least one enhancer, i.e., CMV enhancer. To enhance expression the other elements can be introns (like Promega intron or similar chimeric chicken globin-human immunoglobulin intron).

In the examples below, recombinant AAV9 vectors are described. AAV9 vectors are described, e.g., in U.S. Pat. No. 7,906,111, which is incorporated herein by reference. As used herein, “AAV9 capsid” refers to the AAV9 having the amino acid sequence of GenBank accession: AAS99264 (SEQ ID NO: 29), which is incorporated by reference herein. Some variation from this encoded sequence is encompassed by the present invention, which may include sequences having about 99% identity to the referenced amino acid sequence in GenBank accession:AAS99264 and U.S. Pat. No. 7,906,111 (also WO 2005/033321) (i.e., less than about 1% variation from the referenced sequence), provided that the integrity of the ligand-binding site for the affinity capture purification is maintained and the change in sequences does not substantially alter the pH range for the capsid for the ion exchange resin purification. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

However, other sources of AAV capsids and other viral elements may be selected, as may other immunoglobulin constructs and other vector elements. Methods of generating AAV vectors have been described extensively in the literature and patent documents, including, e.g., WO 2003/042397; WO 2005/033321, WO 2006/110689; U.S. Pat. No. 7,588,772 B2. Suitable AAV may include, e.g, AAV9 [U.S. Pat. No. 7,906,111; US 2011-0236353-A1], rh10 [WO 2003/042397] and/or hu37 [see, e.g., U.S. Pat. No. 7,906,111; US 2011-0236353-A1]. However, other AAV, including, e.g., AAV1, AAV2, AAV3B, AAV4, AAV5, AAV6, AAV7, AAV8 [U.S. Pat. Nos. 7,790,449; 7,282,199], among others be selected for preparing the AAV vectors described herein.

In still other embodiments, another suitable viral vector may be selected. Examples of such vectors may include, e.g., lentivirus, retrovirus, and the like.

### Uses and Regimens

The compositions are designed to administer at least one anti-ebola antibody as provided herein. In one embodiment, the antibody is expressed from a vector (e.g., an AAV). In another embodiment, the antibody is delivered directly to the patient. In still other embodiments, compositions may contain a combination of one or more vectors and/or one or more immunoglobulins. The use of compositions described herein in therapeutic methods are described, as are uses of these compositions in therapies which may optionally involve delivery of one or more other active agents.

As stated above, a composition may contain additional anti-ebola active vectors apart from the rAAV carrying the anti-ebola immunoglobulin cassettes. For example, two or more different AAV may have different expression cassettes which express immunoglobulin polypeptides which assemble in vivo to form a single active immunoglobulin construct.

The compositions can be formulated in dosage units to contain the rAAV, such that each vector stock is present in an amount about 1×10⁹ genome copies (GC) to about 5×10¹³ GC (to treat an average subject of 70 kg in body weight). In one example, the vector concentration is about 3×10¹³ GC, but other amounts such as about 1×10⁹ GC, about 5×10⁹ GC, about 1×10¹⁰ GC, about 5×10¹⁰ GC, about 1×10¹¹ GC, about 5×10¹¹ GC, about 1×10¹² GC, about 5×10¹² GC, or about 1.0×10¹³ GC. Optionally, the rAAV is present in excess of the rAAV stock with the immunoglobulin expression cassette, e.g., about 10:1 to 1.5:1, or about 5:1 to about 3:1, or about 2:1. However, the ratio of first rAAV stock with the transcription factor to rAAV stock with the immunoglobulin may be about 1:1. In certain embodiments, there may be an excess of rAAV.Ab.

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies are the quantitative-PCR (qPCR), particularly the optimized qPCR or digital droplet PCR [Lock Martin, et al, Human Gene Therapy Methods. April 2014, 25 (2): 115-125. doi:10.1089/hgtb.2013.131, published online ahead of editing Dec. 13, 2013].

The rAAV, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, maltose, and water. The selection of the carrier is not a limitation of the present invention. Optionally, the compositions of the invention may contain, in addition to the rAAV and carrier(s), other conventional pharmaceutical ingredients, such as preservatives, or chemical stabilizers.

Any suitable route of administration for the vector composition may be selected, including, e.g., systemic, intravenous, intraperitoneal, subcutaneous, intrathecal, intraocular (e.g., intravitreal), or intramuscular administration.

In another embodiment, a composition may contain each rAAV stock in an amount of about 1.0×10⁸ genome copies (GC)/kilogram (kg) to about 1.0×10¹⁴ GC/kg, and preferably 1.0×10¹¹ GC/kg to 1.0×10¹³ GC/kg to a human patient. Preferably, each rAAV stock is administered in an amount of about 1.0×10⁸ GC/kg, 5.0×10⁸ GC/kg, 1.0×10⁹ GC/kg, 5.0×10⁹ GC/kg, 1.0×10¹⁰ GC/kg, 5.0×10¹⁰ GC/kg, 1.0×10¹¹ GC/kg, 5.0×10¹¹ GC/kg, or 1.0×10¹² GC/kg, 5.0×10¹² GC/kg, 1.0×10¹³ GC/kg, 5.0×10¹³ GC/kg, 1.0×10¹⁴ GC/kg.

When packaged in two or more viral stocks, the replication-defective rAAV compositions are preferably administered simultaneously. However, the viral stocks may be delivered.

In one embodiment, the rAAV compositions may be delivered systemically, directly to a target tissue or organ (e.g., lung, liver), intranasally, subcutaneously, or by another suitable route.

The following examples are illustrative only.

### EXAMPLES

### Example 1: Construction of Humanized Anti-Ebola Antibodies

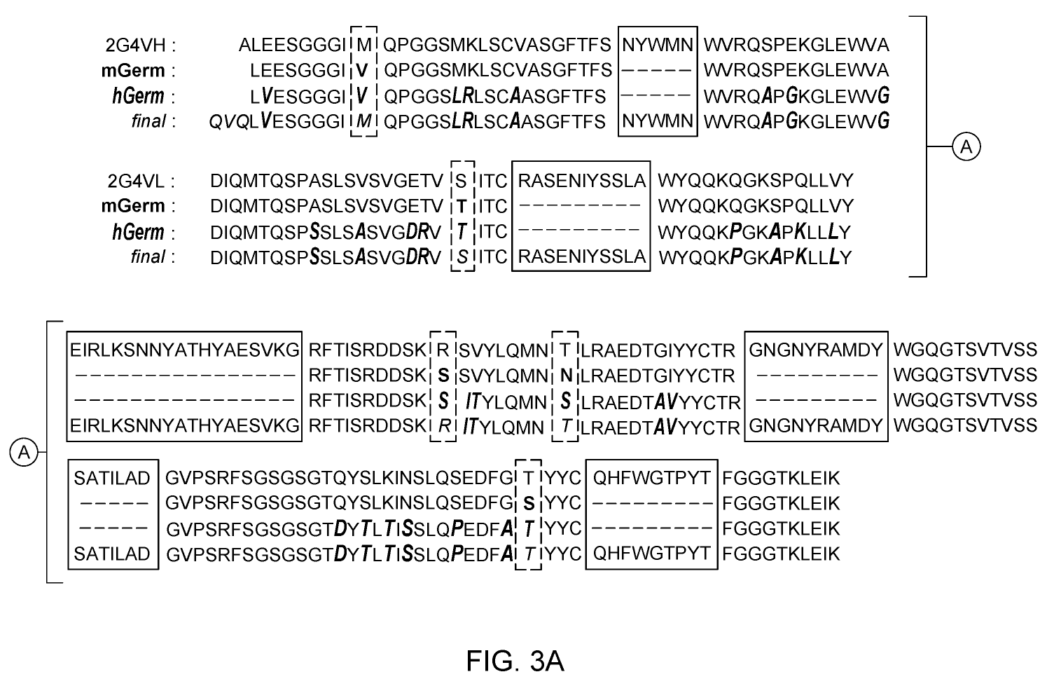

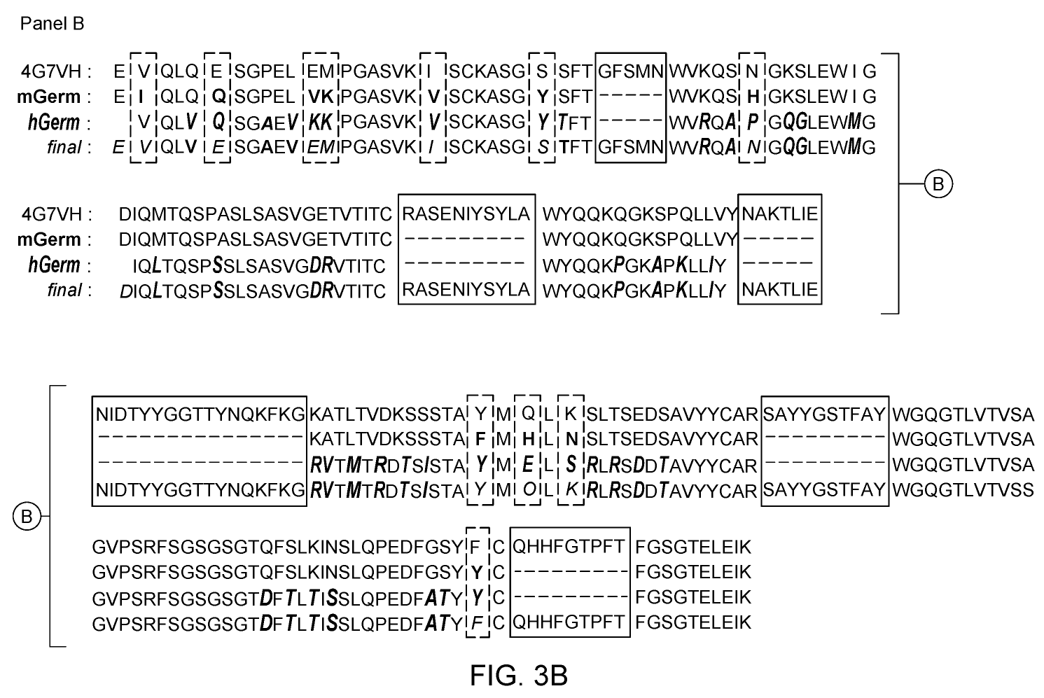

In FIG. 3A, the Kabat nomenclature was used to identify the CDRs of heavy and light chain in mouse 2G4 antibody. Closest mouse immunoglobulin germline sequences were identified by IgBLAST search for closest homology to the 2G4 antibody. [Audet J. et al, Sci Rep, 2014 Nov. 6; 4: 6881, pp 1-8]. Human immunoglobulin germline sequences corresponding to the 2G4 heavy and light variable domains were determined by IgBLAST using the mouse 2G4 amino acid sequences as the input. [Audet J. et al, Sci Rep, 2014 Nov. 6; 4: 6881, pp 1-8]. Human and mouse germline sequences were aligned with the 2G4 amino acid sequences and somatic mutations from the mouse germline sequence were identified in the 2G4 mouse framework regions. Somatic mutations were incorporated into human germline sequences in corresponding framework positions. CDRs from the original mouse 2G4 sequences were grafted onto the corresponding locations on the modified human germline sequence to construct the final humanized variable region sequences.

In FIG. 3B, the Kabat nomenclature scheme was used to identify the CDRs of heavy and light chain in mouse 4G7 antibody. Closest mouse immunoglobulin germline sequences were identified by IgBLAST search for closest homology to the 4G7 antibody. [Audet, 2014 cited above]. Human immunoglobulin germline sequences corresponding to the 4G7 heavy and light variable domains were determined by IgBLAST using the mouse 4G7 amino acid sequences as the input. Human and mouse germline sequences were aligned with the 4G7 amino acid sequences and somatic mutations from the mouse germline sequence were identified in the 4G7 mouse framework regions [Audet, 2014, cited above]. Somatic mutations were incorporated into human germline sequences in corresponding framework positions. CDRs from the original mouse 4G7 sequences were grafted onto the corresponding locations on the modified human germline sequence to construct the final humanized variable region sequences.

Boxes with dashed lines on FIGS. 3A and 3B indicate the locations of the somatic mutations that were transferred to the human frameworks. Boxes with solid lines indicate the positions of the mouse CDR regions that were grafted onto human frameworks.

### Somatic Mutations Changes to Human Germline Sequences

| | 2G4VH | 2G4VL | 4G7VH | 4G7VL |
|---|---|---|---|---|
| Framework 1 | V12M | T20S | V2V, Q6E, K12E, | |
| | | | K13M, V20I, Y27S | |
| Framework 2 | | | P41N | |
| Framework 3 | S79R, N87T | T85T | Y80Y, E82Q, S84K | Y87F |
| Framework 4 | | | | |

### Example 2

The genes encoding the murine antibodies 4G7, 2G4, and the humanized antibody c13C6 were cloned into AAV9 vectors to provide a more efficient and practical method of manufacturing antibodies against EBOV for delivery to humans.

### A. Materials and Methods

AAV Vectors

AAV9 vectors expressing the heavy and light chains of 2G4, 4G7 or c13C6 monoclonal antibodies (mAbs) under the control of a hybrid cytomegalovirus enhancer chicken β-actin promoter were constructed and produced.

Expression Assay

Expression of antibody in serum, bronchoalveolar lavage fluid (BALF) and nasal lavage fluid (NLF) was detected using a protein A ELISA as previously described [Greig, J. A., et al. PLoS One 2014, 9 (11), e112268].

Intramuscular Dosing of AAV9 Vector in Mice

Female, 6-8 week old, BALB/c or BALB/c Rag mice were purchased from the Jackson Laboratory and housed at the Animal Facility of the Translational Research Laboratories at the University of Pennsylvania. Mice were anesthetized by intraperitoneal injection of ketamine/xylazine. The area of the limb to be injected was prepped with 70% ethanol and the approximate external region of the gastrocnemius muscle identified visually. Using a Hamilton syringe (with a 50 μl capacity), AAV9 vector(s) diluted in PBS to a total volume of 40 μl was injected directly into this muscle group through the skin. All animal procedures were approved by the Institutional Animal Care Committee of the University of Pennsylvania.

Ebola Virus (EBOV) Systemic Challenge Experiments in Mice

All mouse challenge studies occurred 14 days after AAV9 vector administration. Mice were anesthetized with inhalational isoflurane (Baxter Healthcare) and challenged by an intraperitoneal injection of 100 μl of 1,000 LD50 of the MA-ZEBOV strain Mayanja. Body weight and clinical signs were recorded daily for 28 days post-challenge. On day 28 post-challenge, mice were sacrificed. All work was performed in the Biosafety Level 4 facility at NML, PHAC. All animal procedures and scoring sheets were approved by the Institutional Animal Care Committee at the NML of the PHAC according to the guidelines of the Canadian Council on Animal Care.

Intranasal Dosing of AAV9 Vector Dosing in Mice

Female, 6-8 week old, BALB/c mice were purchased from the Jackson Laboratory and housed at the Animal Facility of the Translational Research Laboratories at the University of Pennsylvania. Mice were anesthetized by intraperitoneal injection of ketamine/xylazine and then suspended by their dorsal incisors. The mice received AAV9 vector(s) diluted in PBS to a total volume of 50 μl. The area of the limb to be injected was prepped with 70% ethanol and the approximate external region of the gastrocnemius muscle identified visually. Using a Hamilton syringe (with a 50 μl capacity), AAV9 vector(s) diluted in PBS to a total volume of 40 μl was injected directly into this muscle group through the skin. All animal procedures were approved by the Institutional Animal Care and Use Committee of the University of Pennsylvania.

Ebola Virus (EBOV) Nasal Challenge Experiments in Mice

All mouse challenge studies occurred 14 days after AAV9 vector administration. Mice were anesthetized with inhalational isoflurane (Baxter Healthcare) and challenged by intranasal inoculation of 50 μl of 1,000 LD50 (median lethal dose) of the MA-ZEBOV strain Maying a (1.29×10⁷ focus forming units/mL), which was obtained from Mike Bray (NIAID). Body weight and clinical signs were recorded daily for 28 days post-challenge. On day 28 post-challenge, mice were sacrificed. All work was performed in the Biosafety Level 4 facility at NML, PHAC. All animal procedures and scoring sheets were approved by the Institutional Animal Care Committee at the National Microbiology Laboratory (NML) of the Public Health Agency of Canada (PHAC) according to the guidelines of the Canadian Council on Animal Care.

Statistical Analysis

The difference of the survival curves between two groups was tested using the log-rank test implemented in the “survival” package in R language (www.r-project.org). The log-rank test compares estimates of the hazard functions of the two groups at each observed event time. The test statistic is constructed by computing the observed and expected number of events in one of the groups at each observed event time and then adding these to obtain an overall summary across all-time points where there is an event. A test was considered significant when the P value was less than 0.05.

### B. Results and Discussion

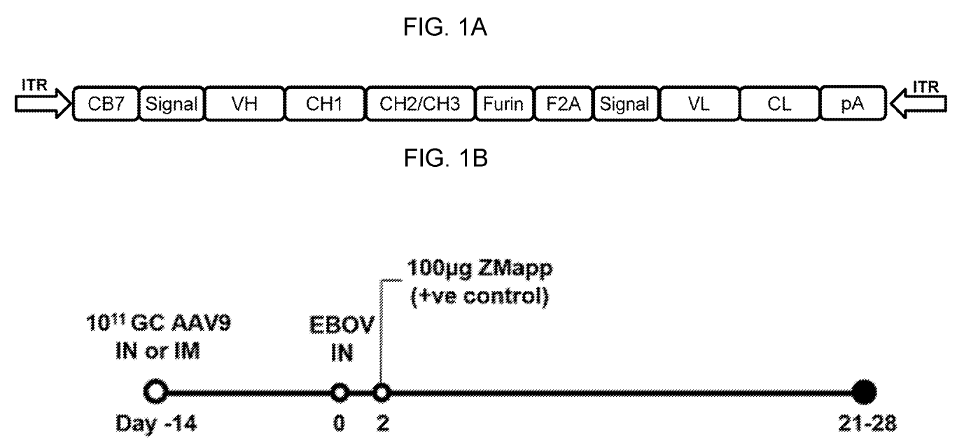

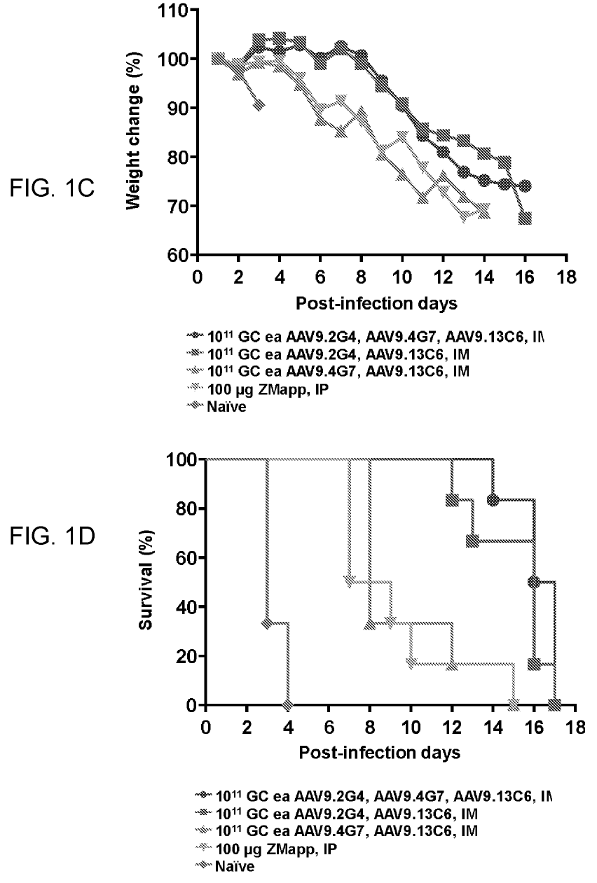

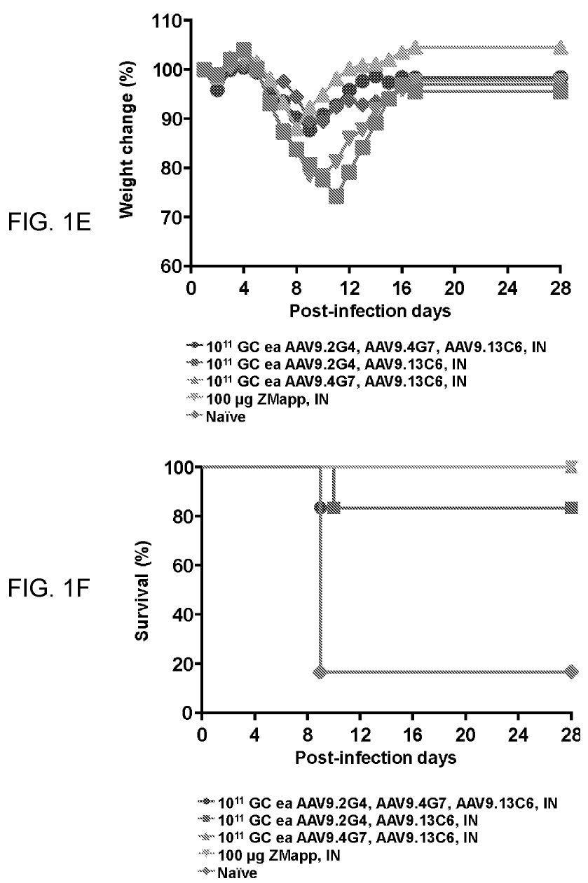

To confer protection against EBOV infection high levels of systemic-circulating binding EBOV antibodies are needed. A mixture of three different AAV9 vectors expressing 4G7, 2G4 and c13C6 Abs (1×10¹¹ GC each) under the transcriptional control of the CB7 promoter (FIG. 1A) were mixed and injected IM in BALB/c mice. Since new studies had demonstrated that a two-mAb cocktail [Qiu X, Audet J, Lv M, et al. Two-mAb cocktail protects macaques against the Makona variant of Ebola virus. Sci Transl Med 2016; 8:329ra33] was as effective as the three mAb cocktail we also evaluated the protective efficacy of a mixture of AAV9 vectors expressing either 4G7 and c13C6 Abs or 2G4 and c13C6 (1×10¹¹ GC each) to determine which combination of mAbs was more effective in the context of AAV prophylaxis (study schematic presented in FIG. 1B). Following IM injection of the AAV vectors into immunocompetent (BALB/c) mice, the potency of the protective efficacy of all three mAbs was hampered by systemic B cell-specific immune responses raised against the mouse/human chimeric mAbs (data not shown) resulting in ineffective protection against a lethal challenge with 1,000 LD50 mouse-adaptors (MA)-EBOV (FIGS. 1C and 1D). Interestingly, when the AAV vectors were given to mice IN (FIGS. 1E and 1 F) we achieved full protection against airway EBOV challenge with similar kinetics of weight loss to ZMapp. The studies demonstrated that a two-AAV.mAb cocktail (2G4 and c13C6) were effective at conferring ˜83% survival with similar weight loss to ZMapp. This two mAb cocktail was developed for effective prophylaxis against EBOV infection.

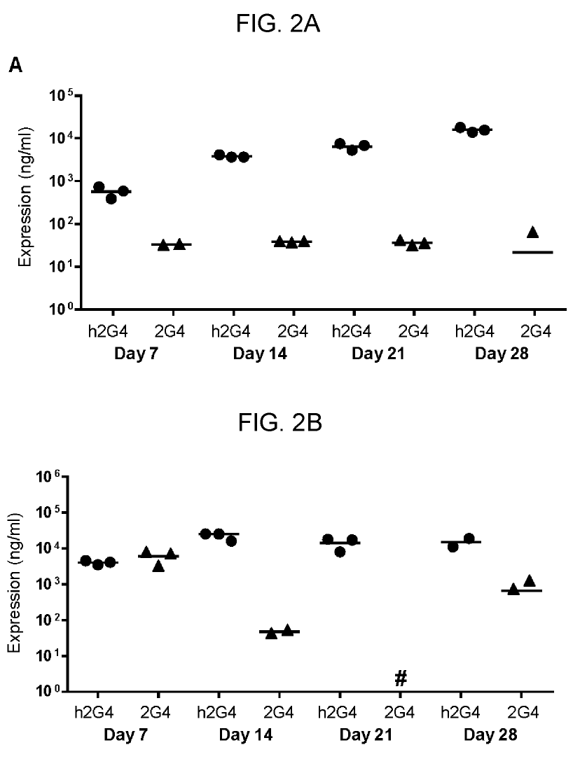

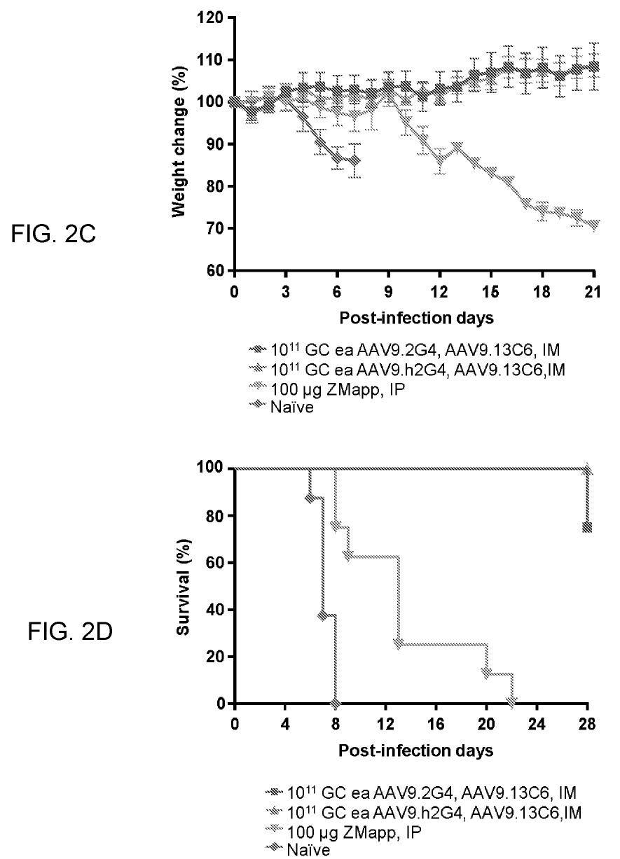

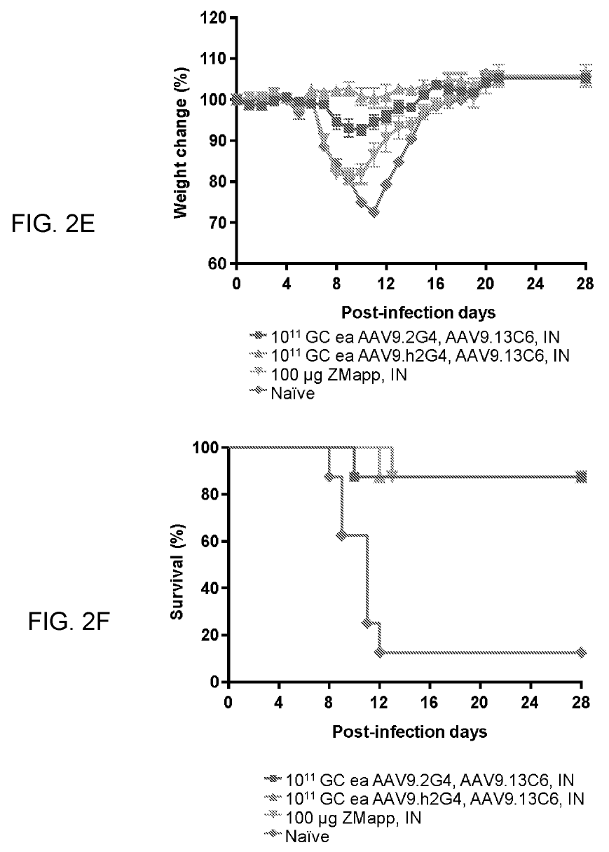

To improve on the safety of 2G4 for human use we humanized the mAb by aligning the human and mouse germline sequences with the 2G4 amino acid sequences. Somatic mutations were incorporated into the human germline sequences in corresponding framework positions. CDRs from the original mouse 2G4 sequences were then grafted onto the corresponding locations on the modified human germline sequence to construct the final humanized variable region sequences. AAV9 vectors were constructed to express the humanized 2G4 (noted as h2G4) and expression tested in BALB/c mice following IN or IM delivery of 1×10¹¹ GC of AAV9 vectors. Interestingly, humanization of 2G4 vastly improved its expression profile in the serum of IN and IM injected mice (FIGS. 2A to 2D). For IN delivery, expression of AAV9.h2G4 was at least 2 logs improved over that of AAV9.2G4. The most impressive difference was observed in the IM setting in which AAV9.h2G4 resulted in high level, and sustained gene expression for the duration of this study (28 days). The expression profile of Ab was also assessed in the serum, the bronchoalveolar lavage fluid (BALF) and the nasal lavage fluid (NLF) of BALB/c mice fourteen days after IN delivery 1×10¹¹ GC of AAV9.2G4 or AAV9.h2G4. In both serum and BALF we observed a 2-fold increase in the level of Ab expression when using AAV9.h2G4 (9265 ng/ml in serum; 3617 ng/ml in BALF) compared to AAV9.2G4 (5117 ng/ml in serum; 1549 ng/ml in BALF). The marked improvement in expression by the AAV9.h2G4 vector was evident in the NLF with 34 ng/ml for AAV9.h2G4 versus undetectable levels for AAV9.2G4. To avoid anti-transgene B cell responses that could compromise the efficacy of the IM AAV9 delivery, immunodeficient BALB/c Rag mice were given IM a mixture of either AAV9.h2G4 and AAV9.c13C6 vectors, or the AAV9.2G4 (non-humanized) and AAV9.c13C6 vectors (FIGS. 2B and 2C). Naive mice succumbed to the EBOV infection by day 8, and mice given a single administration of ZMapp once at day 2 post the challenge succumbed to the infection by day 22. Interestingly, mice given a mixture of either AAV9.h2G4 and AAV9.c13C6 vectors, or the AAV9.2G4 and AAV9.c13C6 vectors survived the EBOV challenge which was accompanied by no significant weight loss. For the group of mice given the mixture of AAV9.2G4 and AAV9.c13C6 vectors one mouse was found dead at day 28 with no apparent disease or viral burden. The impact of the humanization of 2G4 became apparent following IN delivery of a mixture of either AAV9.h2G4 and AAV9.c13C6 vectors, or the AAV9.2G4 and AAV9.c13C6 vectors. In this setting both vector regimens fared better against the EBOV challenge than that of ZMapp and impressively, the mixture of AAV9.h2G4 and AAV9.c13C6 vectors resulted in no weight loss and 87.5% survival (FIGS. 2E and 2F). Notably, despite significant weight loss for both ZMapp and the mixture of AAV9.2G4 and AAV9.c13C6 vectors both regimens also conferred an 87.5% survival against IN challenge with EBOV.

The utility of ZMapp is marred by the need for high amounts of product to treat EBOV infected patients. Further, its limited supply may compromise effective dissemination of product to treat infected subjects in outbreak zones. In a mouse model of EBOV infection, the prophylactic capacity of AAV vectors expressing the components of ZMapp to protect against two different modes of challenge, systemic and airway, with EBOV was provided. Typically, human subjects are treated with ZMapp at the onset of symptom presentation with reports demonstrating that the level of symptom severity impacts the effectiveness of the ZMapp treatment. Given the devastating impact of the recent 2014 EBOV outbreak in West Africa, which claimed the lives of more than 11,000 patients and health workers [Dzau, V. J. & Sands, P. Beyond the Ebola Battle—Winning the War against Future Epidemics. N Engl J Med 2016], prophylaxis against EBOV infection is warranted in areas with active outbreaks.

In conclusion, since AAV-mediated prophylaxis is conferred within days of administration [Limberis, M. P., et al, Sci Transl Med 2013, 5 (187), 187ra172] a single administration of AAV via an injection into the muscle or via non-invasive instillation in the nose is an effective measure to control and contain rapidly spreading infectious virus dissemination in closed communities. The effectiveness of intranasal AAV9 delivery of anti-EBOV antibodies may prove to be important if natural evolution of the virus enhances its ability to be transmitted via a respiratory route [Petrosillo, N., et al. BMC Infect Dis 2015, 15, 43215] or if the virus is weaponized.

### Sequence Listing Free Text The following information is provided for sequences containing free text under numeric identifier <223>.

| SEQ ID | |
|---|---|
| NO: (containing | |
| free text) | Free text under <223> |
| 1 | <223> 4q7sc1h |
| | <220> |
| | <221> misc_feature |
| | <222> (4) . . . (75) |
| | <223> F2a linker |
| | <220> |
| | <221> misc_feature |
| | <222> (76) . . . (135) |
| | <223> leader |
| | <220> |
| | <221> CDS |
| | <222> (136) . . . (489) |
| | <223> 4q7sc1h |
| 2 | <223> Synthetic Construct |
| 3 | <223> 4g7sc1L |
| | <220> |
| | <221> CDS |
| | <222> (1) . . . (321) |
| 4 | <223> Synthetic Construct |
| 5 | <223> 2g4sc1h |
| | <220> |
| | <221> misc_feature |
| | <222> (4) . . . (75) |
| | <223> f2a |
| | <220> |
| | <221> misc_feature |
| | <222> (76) . . . (135) |
| | <223> leader |
| | <220> |
| | <221> CDS |
| | <222> (136) . . . (495) |
| | <223> 2g4sc1h |
| 6 | <223> Synthetic Construct |
| 7 | <223> 2g4sc11 |
| | <220> |
| | <221> CDS |
| | <222> (1) . . . (321) |
| 8 | <223> Synthetic Construct |
| 9 | <223> CB72g4sc1 |
| | <220> |
| | <221> repeat_region |
| | <222> (1) . . . (130) |
| | <223> 5′ ITR |
| | <220> |
| | <221> promoter |
| | <222> (198) . . . (579) |
| | <223> CMV IE promoter |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> misc_feature |
| | <222> (1999) . . . (2055) |
| | <223> leader |
| | <220> |
| | <221> misc_feature |
| | <222> (2056) . . . (2382) |
| | <223> 2g4sc1 light |
| | <220> |
| | <221> CDS |
| | <222> (2377) . . . (2697) |
| | <223> CL |
| | <220> |
| | <221> misc_feature |
| | <222> (2698) . . . (2709) |
| | <223> furin |
| | <220> |
| | <221> misc_feature |
| | <222> (2707) . . . (3207) |
| | <223> 2g4sc1 heavy |
| | <220> |
| | <221> CDS |
| | <222> (3205) . . . (4194) |
| | <223> CH1, CH2-3 |
| | <220> |
| | <221> misc_feature |
| | <222> (3205) . . . (3525) |
| | <223> CH1 |
| | <220> |
| | <221> misc_feature |
| | <222> (3528) . . . (4194) |
| | <223> CH2-3 |
| | <220> |
| | <221> polyA_signal |
| | <222> (4273) . . . (4399) |
| | <223> Rabbit globin poly A |
| | <220> |
| | <221> repeat_region |
| | <222> (4488) . . . (4617) |
| | <223> 3′ ITR |
| 10 | <223> Synthetic Construct |
| 11 | <223> Synthetic Construct |
| 12 | <223> CB74q7sc1 |
| | <220> |
| | <221> repeat_region |
| | <222> (1) . . . (130) |
| | <223> 5′ ITR |
| | <220> |
| | <221> promoter |
| | <222> (198) . . . (579) |
| | <223> CMV IE promoter |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> misc_feature |
| | <222> (1940) . . . (1987) |
| | <223> c-myc 5′ UTR |
| | <220> |
| | <221> misc_feature |
| | <222> (1999) . . . (2055) |
| | <223> leader |
| | <220> |
| | <221> misc_feature |
| | <222> (2056) . . . (2376) |
| | <223> 4q7sc11 |
| | <220> |
| | <221> CDS |
| | <222> (2377) . . . (2697) |
| | <223> CL |
| | <220> |
| | <221> misc_feature |
| | <222> (2698) . . . (2709) |
| | <223> furin |
| | <220> |
| | <221> misc_feature |
| | <222> (2707) . . . (3201) |
| | <223> 2g4sc1heavy |
| | <220> |
| | <221> CDS |
| | <222> (3199) . . . (4188) |
| | <223> CH1, CH2-3 |
| | <220> |
| | <221> misc_feature |
| | <222> (3199) . . . (3519) |
| | <223> CH1 |
| | <220> |
| | <221> misc_feature |
| | <222> (3522) . . . (4188) |
| | <223> CH2-3 |
| | <220> |
| | <221> polyA_signal |
| | <222> (4267) . . . (4393) |
| | <223> rabbit globin polyA |
| | <220> |
| | <221> repeat_region |
| | <222> (4482) . . . (4611) |
| | <223> 3′ ITR |
| 13 | <223> Synthetic Construct |
| 14 | <223> Synthetic Construct |
| 15 | <223> pN3019 TBG 4q7sc1 |
| | <220> |
| | <221> repeat_region |
| | <222> (1) . . . (130) |
| | <223> 5′-ITR |
| | <220> |
| | <221> promoter |
| | <222> (205) . . . (913) |
| | <223> TBG |
| | <220> |
| | <221> TATA_signal |
| | <222> (875) . . . (878) |
| | <220> |
| | <221> Intron |
| | <222> (939) . . . (1071) |
| | <223> SV40 misc intron |
| | <220> |
| | <221> misc_feature |
| | <222> (1160) . . . (1207) |
| | <223> c-myc 5′UTR |
| | <220> |
| | <221> misc_feature |
| | <222> (1219) . . . (1227) |
| | <223> leader |
| | <220> |
| | <221> misc_feature |
| | <222> (1276) . . . (1602) |
| | <223> 4q7 light |
| | <220> |
| | <221> misc_feature |
| | <222> (1597) . . . (1917) |
| | <223> CL |
| | <220> |
| | <221> misc_feature |
| | <222> (1918) . . . (1929) |
| | <223> furin |
| | <220> |
| | <221> misc_feature |
| | <222> (1927) . . . (2421) |
| | <223> 4q7sc1h |
| | <220> |
| | <221> misc_feature |
| | <222> (2419) . . . (2739) |
| | <223> CH1 |
| | <220> |
| | <221> misc_feature |
| | <222> (2742) . . . (3408) |
| | <223> CH2-3 |
| | <220> |
| | <221> polyA_signal |
| | <222> (3527) . . . (3741) |
| | <223> Bovine Growth Hormone polyadenylation |
| | (BGH-PolyA) |
| | <220> |
| | <221> repeat_region |
| | <222> (3829) . . . (3958) |
| | <223> 3′-ITR |
| 16 | <223> TBG 2g4sc1 |
| | <220> |
| | <221> repeat_region |
| | <222> (1) . . . (130) |
| | <223> 5′-ITR |
| | <220> |
| | <221> promoter |
| | <222> (205) . . . (913) |
| | <223> TBG |
| | <220> |
| | <221> TATA_signal |
| | <222> (875) . . . (878) |
| | <220> |
| | <221> Intron |
| | <222> (939) . . . (1071) |
| | <223> SV40 intron |
| | <220> |
| | <221> misc_feature |
| | <222> (1160) . . . (1207) |
| | <223> c-myc 5′UTR |
| | <220> |
| | <221> misc_feature |
| | <222> (1219) . . . (1227) |
| | <223> leader |
| | <220> |
| | <221> misc_feature |
| | <222> (1276) . . . (1596) |
| | <223> 2g4sc1 |
| | <220> |
| | <221> misc_feature |
| | <222> (1597) . . . (1917) |
| | <223> CL |
| | <220> |
| | <221> misc_feature |
| | <222> (1918) . . . (1929) |
| | <223> furin |
| | <220> |
| | <221> misc_feature |
| | <222> (1927) . . . (2427) |
| | <223> 2g4sc1 heavy |
| | <220> |
| | <221> misc_feature |
| | <222> (2425) . . . (2745) |
| | <223> CH1 |
| | <220> |
| | <221> misc_feature |
| | <222> (2748) . . . (3414) |
| | <223> CH2-3 |
| | <220> |
| | <221> polyA_signal |
| | <222> (3533) . . . (3747) |
| | <223> bovine growth hormone polyA |
| | <220> |
| | <221> repeat_region |
| | <222> (3835) . . . (3964) |
| | <223> 3′-ITR |
| 17 | <213> Mus musculus |
| | <220> |
| | <221> misc_feature |
| | <222> (28) . . . (32) |
| | <223> Xaa can be any naturally occurring amino acid |
| | <220> |
| | <221> misc_feature |
| | <222> (47) . . . (65) |
| | <223> Xaa can be any naturally occurring amino acid |
| | <220> |
| | <221> misc_feature |
| | <222> (98) . . . (107) |
| | <223> Xaa can be any naturally occurring amino acid |
| 18 | <213> Homo sapiens |
| | <220> |
| | <221> misc_feature |
| | <222> (28) . . . (32) |
| | <223> Xaa can be any naturally occurring amino acid |
| | <220> |
| | <221> misc_feature |
| | <222> (47) . . . (65) |
| | <223> Xaa can be any naturally occurring amino acid |
| | <220> |
| | <221> misc_feature |
| | <222> (98) . . . (107) |
| | <223> Xaa can be any naturally occurring amino acid |
| 19 | <213> Mus musculus |
| | <220> |
| | <221> misc_feature |
| | <222> (24) . . . (34) |
| | <223> Xaa can be any naturally occurring amino acid |
| | <220> |
| | <221> misc_feature |
| | <222> (50) . . . (56) |
| | <223> Xaa can be any naturally occurring amino acid |
| | <220> |
| | <221> misc_feature |
| | <222> (89) . . . (97) |
| | <223> Xaa can be any naturally occurring amino acid |
| 20 | <213> Homo sapiens |
| | <220> |
| | <221> misc_feature |
| | <222> (24) . . . (34) |
| | <223> Xaa can be any naturally occurring amino acid |
| | <220> |
| | <221> misc_feature |
| | <222> (50) . . . (56) |
| | <223> Xaa can be any naturally occurring amino acid |
| | <220> |
| | <221> misc_feature |
| | <222> (89) . . . (97) |
| | <223> Xaa can be any naturally occurring amino acid |
| 21 | <213> Mus musculus |
| | <220> |
| | <221> misc_feature |
| | <222> (31) . . . (35) |
| | <223> Xaa can be any naturally occurring amino acid |
| | <220> |
| | <221> misc_feature |
| | <222> (50) . . . (66) |
| | <223> Xaa can be any naturally occurring amino acid |
| | <220> |
| | <221> misc_feature |
| | <222> (99) . . . (108) |
| | <223> Xaa can be any naturally occurring amino acid |
| 22 | <213> Homo sapiens |
| | <220> |
| | <221> misc_feature |
| | <222> (30) . . . (34) |
| | <223> Xaa can be any naturally occurring amino acid |
| | <220> |
| | <221> misc_feature |
| | <222> (49) . . . (65) |
| | <223> Xaa can be any naturally occurring amino acid |
| | <220> |
| | <221> misc_feature |
| | <222> (98) . . . (107) |
| | <223> Xaa can be any naturally occurring amino acid |
| 23 | <213> Mus musculus |
| | <220> |
| | <221> misc_feature |
| | <222> (24) . . . (34) |
| | <223> Xaa can be any naturally occurring amino acid |
| | <220> |
| | <221> misc_feature |
| | <222> (50) . . . (56) |
| | <223> Xaa can be any naturally occurring amino acid |
| | <220> |
| | <221> misc_feature |
| | <222> (89) . . . (97) |
| | <223> Xaa can be any naturally occurring amino acid |
| 24 | <213> Homo sapiens |
| | <220> |
| | <221> misc_feature |
| | <222> (23) . . . (33) |
| | <223> Xaa can be any naturally occurring amino acid |
| | <220> |
| | <221> misc_feature |
| | <222> (49) . . . (55) |
| | <223> Xaa can be any naturally occurring amino acid |
| | <220> |
| | <221> misc_feature |
| | <222> (88) . . . (96) |
| | <223> Xaa can be any naturally occurring amino acid |
| 25 | <223> Humanized 2G4VH |
| 26 | <223> Humanized 2G4VL |
| 27 | <223> Humanized 4G7VH |
| 28 | <223> Humanized 4G7VL |
| 29 | <223> capsid protein vp1 (adeno-associated virus 9) |

All publications and references to GenBank and other sequences cited in this specification are incorporated herein by reference. All publications cited in this specification are incorporated herein by reference in their entireties, as is U.S. Provisional Patent Application No. 62/399,362, filed Sep. 24, 2016. Similarly, the Sequence Listing labeled 16-7984PCT_ST25.txt filed herewith is hereby incorporated by reference. While the invention has been described with reference to particularly preferred embodiments, it will be appreciated that modifications can be made without departing from the spirit of the invention. Such modifications are intended to fall within the scope of the appended claims.

1. A recombinant vector which comprises an expression cassette comprising the nucleic acid sequence encoding a humanized anti-ebola antibody under the control of regulatory sequences which direct expression of the antibody in target cells, wherein the anti-ebola antibody is selected from:
(a) a humanized 2G4 anti-ebola antibody (H2G4) comprising:
(i) a heavy chain comprising a variable region having the amino acid sequence of SEQ ID NO: 6 (H2G4VH); and
(ii) a light chain comprising a variable region having the amino acid sequence of SEQ ID NO: 8 (H2G4VL); or
(b) a humanized 4G7 anti-ebola antibody (H4G7) comprising:
(i) a heavy chain comprising a variable region having the amino acid sequence of SEQ ID NO: 2 (H4G7VH); and
(ii) a light chain comprising a variable region having the amino acid sequence of SEQ ID NO: 4 (H4G7VL).

2. The recombinant vector according to claim 1, wherein the antibody is the H2G4, and wherein the heavy chain further comprises the constant regions of SEQ ID NO: 11.

3. The recombinant vector according to claim 1, wherein the antibody is the H4G7, and wherein the heavy chain further comprises the constant domain of SEQ ID NO: 14.

4. The recombinant vector according to claim 1, wherein the antibody is the H2G4, and wherein the light chain further comprises the constant domain of SEQ ID NO: 10.

5. The recombinant vector according to claim 1, wherein the antibody is the H4G7, and wherein the light chain further comprises the constant domain of SEQ ID NO: 13.

6. The recombinant vector according to claim 1, wherein the antibody is selected from a full-length antibody, an immunoadhesin, a bispecific antibody and an scFV fragment.

7. The recombinant vector according to claim 1, wherein the vector is a recombinant adeno-associated virus (rAAV).

8. The recombinant vector according to claim 7, wherein the rAAV has an AAV9 capsid.

9. A composition comprising a carrier, diluent, excipient and/or preservative and a recombinant vector according to claim 1.

10. The composition according to claim 9, wherein the composition is a liquid suspension.

11. The composition according to claim 9, wherein the composition further comprises a second anti-ebola active component.

12. The composition according to claim 11, wherein the composition further comprises an anti-ebola antibody.

13. The composition according to claim 12, wherein the anti-ebola antibody is the same as that expressed from the recombinant vector.

14. The composition according to claim 12, wherein the anti-ebola antibody is different from that expressed from the recombinant vector.

15. A method for preventing ebola infection or improving survival rates against ebola in a human population comprising delivering an effective amount of a recombinant vector to a subject at risk of infection, wherein the recombinant vector which comprises an expression cassette comprising the nucleic acid sequence encoding a humanized anti-ebola antibody under the control of regulatory sequences which direct expression of the antibody in target cells, wherein the anti-ebola antibody is selected from:
(a) a humanized 2G4 anti-ebola antibody (H2G4) comprising:
(i) a heavy chain comprising a variable region having the amino acid sequence of SEQ ID NO: 6 (H2G4VH); and
(ii) a light chain comprising a variable region having the amino acid sequence of SEQ ID NO: 8 (H2G4VL); or
(b) a humanized 4G7 anti-ebola antibody (H4G7) comprising:
(i) a heavy chain comprising a variable region having the amino acid sequence of SEQ ID NO: 2 (H4G7VH); and
(ii) a light chain comprising a variable region having the amino acid sequence of SEQ ID NO: 4 (H4G7VL).

16. The method according to claim 15, wherein the method further comprises delivering at least a second anti-ebola antibody.

17. The method according to clam 16, wherein the second anti-ebola antibody is the same as that expressed from the recombinant vector, or wherein the second anti-ebola antibody is different from that expressed from the recombinant vector.

18. (canceled)

19. The method according to claim 16, wherein the recombinant vector and the second anti-ebola antibody are delivered to the subject via different routes of administration.

20. The method according to claim 16, wherein the second anti-ebola antibody is delivered intravenously, intramuscularly, or subcutaneously, and wherein the recombinant vector is delivered intranasally.

21-28. (canceled)

29. A recombinant humanized antibody which is useful in preventing infection with ebola virus, said antibody selected from:
(a) a humanized 2G4 anti-ebola antibody (H2G4) comprising:
(i) a heavy chain comprising a variable region having the amino acid sequence of SEQ ID NO: 6 (H2G4VH); and
(ii) a light chain comprising a variable region having the amino acid sequence of SEQ ID NO: 8 (H2G4VL); or
(b) a humanized 4G7 anti-ebola antibody (H4G7) comprising:
(i) a heavy chain comprising a variable region having the amino acid sequence of SEQ ID NO: 2 (H4G7VH); and
(ii) a light chain comprising a variable region having the amino acid sequence of SEQ ID NO: 4 (H4G7VL).

30-70. (canceled)


SEQUENCE LISTING (29 sequences)

SEQ ID NO: 1 - DNA, 495 bp, Artificial Sequence
  4q7sc1h
  misc_feature (4)..(75): F2a linker
  misc_feature (76)..(135): leader
  CDS (136)..(489): 4q7sc1h
cgggccccagtgaagcagaccctgaacttcgatctgctgaagctggccggagatgtggagagcaacccaggaccaatgtacagaatgcagctgctgagctgcatcgccctgagcctggccctggtgaccaacagcgaggtgcagctgcaggagagcggaccagagctggagatgccaggagccagcgtgaagatcagctgcaaggccagcggaagcagcttcaccggattcagcatgaactgggtgaagcagagcaacggaaagagcctggagtggatcggaaacatcgatacctactacggaggaaccacctacaaccagaagttcaagggaaaggccaccctgaccgtggataagagcagcagcaccgcctacatgcagctgaagagcctgaccagcgaggatagcgccgtgtactactgcgcccggagcgcctactacggaagcaccttcgcctactggggacagggaaccctggtgaccgtgagcagcgct

SEQ ID NO: 2 - protein, 118 aa, Artificial Sequence
  Synthetic Construct
EVQLQESGPELEMPGASVKISCKASGSSFTGFSMNWVKQSNGKSLEWIGNIDTYYGGTTYNQKFKGKATLTVDKSSSTAYMQLKSLTSEDSAVYYCARSAYYGSTFAYWGQGTLVTVS

SEQ ID NO: 3 - DNA, 321 bp, Artificial Sequence
  4g7sc1L
  CDS (1)..(321)
gatatccagatgacccagagcccagccagcctgagcgccagcgtgggagagaccgtgaccatcacctgccgggccagcgagaacatctacagctacctggcctggtaccagcagaagcagggaaagagcccacagctgctggtgtacaacgccaagaccctgatcgagggagtgccaagccggttcagcggaagcggaagcggaacccagttcagcctgaagatcaacagcctgcagccagaggatttcggaagctacttctgccagcaccacttcggaaccccattcaccttcggaagcggaaccgagctggagatcaag

SEQ ID NO: 4 - protein, 107 aa, Artificial Sequence
  Synthetic Construct
DIQMTQSPASLSASVGETVTITCRASENIYSYLAWYQQKQGKSPQLLVYNAKTLIEGVPSRFSGSGSGTQFSLKINSLQPEDFGSYFCQHHFGTPFTFGSGTELEIK

SEQ ID NO: 5 - DNA, 501 bp, Artificial Sequence
  2g4sc1h
  misc_feature (4)..(75): f2a
  misc_feature (76)..(135): leader
  CDS (136)..(495): 2g4sc1h
cgggccccagtgaagcagaccctgaacttcgatctgctgaagctggccggagatgtggagagcaacccaggaccaatgtacagaatgcagctgctgagctgcatcgccctgagcctggccctggtgaccaacagccaggtggccctggaggagagcggaggaggactgatgcagccaggaggaagcatgaagctgagctgcgtggccagcggattcaccttcagcaactactggatgaactgggtgcggcagagcccagagaagggactggagtgggtggccgagatccggctgaagagcaacaactacgccacccactacgccgagagcgtgaagggacggttcaccatcagccgggatgatagcaagcggagcgtgtacctgcagatgaacaccctgcgggccgaggataccggaatctactactgcacccggggaaacggaaactaccgggccatggattactggggacagggaaccagcgtgaccgtgagcagcgct

SEQ ID NO: 6 - protein, 120 aa, Artificial Sequence
  Synthetic Construct
QVALEESGGGLMQPGGSMKLSCVASGFTFSNYWMNWVRQSPEKGLEWVAEIRLKSNNYATHYAESVKGRFTISRDDSKRSVYLQMNTLRAEDTGIYYCTRGNGNYRAMDYWGQGTSVTVS

SEQ ID NO: 7 - DNA, 321 bp, Artificial Sequence
  2g4sc1l
  CDS (1)..(321)
gatatccagatgacccagagcccagccagcctgagcgtgagcgtgggagagaccgtgagcatcacctgccgggccagcgagaacatctacagcagcctggcctggtaccagcagaagcagggaaagagcccacagctgctggtgtacagcgccaccatcctggccgatggagtgccaagccggttcagcggaagcggaagcggaacccagtacagcctgaagatcaacagcctgcagagcgaggatttcggaacctactactgccagcacttctggggaaccccatacaccttcggaggaggaaccaagctggagatcaag

SEQ ID NO: 8 - protein, 107 aa, Artificial Sequence
  Synthetic Construct
DIQMTQSPASLSVSVGETVSITCRASENIYSSLAWYQQKQGKSPQLLVYSATILADGVPSRFSGSGSGTQYSLKINSLQSEDFGTYYCQHFWGTPYTFGGGTKLEIK

SEQ ID NO: 9 - DNA, 7438 bp, Artificial Sequence
  CB72g4sc1
  repeat_region (1)..(130): 5′ ITR
  promoter (198)..(579): CMV IE promoter
  promoter (582)..(862): CB promoter
  Intron (956)..(1928): chicken beta-actin intron
  misc_feature (1999)..(2055): leader
  misc_feature (2056)..(2382): 2g4sc1 light
  CDS (2377)..(2697): CL
  misc_feature (2698)..(2709): furin
  misc_feature (2707)..(3207): 2g4sc1 heavy
  CDS (3205)..(4194): CH1, CH2-3
  misc_feature (3205)..(3525): CH1
  misc_feature (3528)..(4194): CH2-3
  polyA_signal (4273)..(4399): Rabbit globin poly A
  repeat_region (4488)..(4617): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctgccgccccaccatgcgcatgcagctgctgctgctgatcgccctgagcctggccctggtgaccaacagcgatatccagatgacccagagcccagccagcctgagcgtgagcgtgggagagaccgtgagcatcacctgccgggccagcgagaacatctacagcagcctggcctggtaccagcagaagcagggaaagagcccacagctgctggtgtacagcgccaccatcctggccgatggagtgccaagccggttcagcggaagcggaagcggaacccagtacagcctgaagatcaacagcctgcagagcgaggatttcggaacctactactgccagcacttctggggaaccccatacaccttcggaggaggaaccaagctggagatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgccggaagcggcgggccccagtgaagcagaccctgaacttcgatctgctgaagctggccggagatgtggagagcaacccaggaccaatgtacagaatgcagctgctgagctgcatcgccctgagcctggccctggtgaccaacagccaggtggccctggaggagagcggaggaggactgatgcagccaggaggaagcatgaagctgagctgcgtggccagcggattcaccttcagcaactactggatgaactgggtgcggcagagcccagagaagggactggagtgggtggccgagatccggctgaagagcaacaactacgccacccactacgccgagagcgtgaagggacggttcaccatcagccgggatgatagcaagcggagcgtgtacctgcagatgaacaccctgcgggccgaggataccggaatctactactgcacccggggaaacggaaactaccgggccatggattactggggacagggaaccagcgtgaccgtgagcagcgctagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtatagcctgagcagcgtggtgaccgtgccaagcagcagcctgggaacccagacctacatctgcaacgtgaaccacaagccaagcaacaccaaggtggataagaaggtggagccaaagagctgcgataagacccacacgtgccctccatgtccagcccccgaactgctgggcgggcctagcgtgttcctgtttccccctaagcctaaagatacactgatgattagtagaaccccagaggtcacatgcgtggtcgtggacgtgtcccacgaagagcctgacgtgaagttcaactggtacgtggatggcgtggaggtgcacaatgctaagactaaaccacgcgaagagcagtataatagtacataccgagtcgtgtcagtcctgacagtgctgcaccaggattggctgaacggcaaggagtataagtgcaaggtgtctaacaaggccctgcccgcccctatcgagaaaacaattagcaaggccaaagggcagccacgggaaccccaggtctacactctgccaccctcaagagatgaactgactaagaaccaggtcagcctgacctgtctggtgaaaggcttctaccccagcgacatcgccgtggagtgggaaagtaacggccagcctgagaataactacaagactacccctccagtgctggatagcgacgggtccttcttcctgtatagcaagctgacagtggacaaatcccgctggcagcagggaaacgtcttttcctgttctgtgatgcatgaggccctgcacaatcattacacccagaagagtctgtcactgagccccggcaaatgataagcttgcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 10 - protein, 107 aa, Artificial Sequence [sequence omitted]

SEQ ID NO: 11 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEEPDVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 7432 bp, Artificial Sequence
  CB74q7sc1
  repeat_region (1)..(130): 5′ ITR
  promoter (198)..(579): CMV IE promoter
  promoter (582)..(862): CB promoter
  Intron (956)..(1928): chicken beta-actin intron
  misc_feature (1940)..(1987): c-myc 5′ UTR
  misc_feature (1999)..(2055): leader
  misc_feature (2056)..(2376): 4q7sc1l
  CDS (2377)..(2697): CL
  misc_feature (2698)..(2709): furin
  misc_feature (2707)..(3201): 2g4sc1heavy
  CDS (3199)..(4188): CH1, CH2-3
  misc_feature (3199)..(3519): CH1
  misc_feature (3522)..(4188): CH2-3
  polyA_signal (4267)..(4393): rabbit globin polyA
  repeat_region (4482)..(4611): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctgccgccccaccatgcgcatgcagctgctgctgctgatcgccctgagcctggccctggtgaccaacagcgatatccagatgacccagagcccagccagcctgagcgccagcgtgggagagaccgtgaccatcacctgccgggccagcgagaacatctacagctacctggcctggtaccagcagaagcagggaaagagcccacagctgctggtgtacaacgccaagaccctgatcgagggagtgccaagccggttcagcggaagcggaagcggaacccagttcagcctgaagatcaacagcctgcagccagaggatttcggaagctacttctgccagcaccacttcggaaccccattcaccttcggaagcggaaccgagctggagatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgccggaagcggcgggccccagtgaagcagaccctgaacttcgatctgctgaagctggccggagatgtggagagcaacccaggaccaatgtacagaatgcagctgctgagctgcatcgccctgagcctggccctggtgaccaacagcgaggtgcagctgcaggagagcggaccagagctggagatgccaggagccagcgtgaagatcagctgcaaggccagcggaagcagcttcaccggattcagcatgaactgggtgaagcagagcaacggaaagagcctggagtggatcggaaacatcgatacctactacggaggaaccacctacaaccagaagttcaagggaaaggccaccctgaccgtggataagagcagcagcaccgcctacatgcagctgaagagcctgaccagcgaggatagcgccgtgtactactgcgcccggagcgcctactacggaagcaccttcgcctactggggacagggaaccctggtgaccgtgagcagcgctagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtatagcctgagcagcgtggtgaccgtgccaagcagcagcctgggaacccagacctacatctgcaacgtgaaccacaagccaagcaacaccaaggtggataagaaggtggagccaaagagctgcgataagacccacacgtgccctccatgtccagcccccgaactgctgggcgggcctagcgtgttcctgtttccccctaagcctaaagatacactgatgattagtagaaccccagaggtcacatgcgtggtcgtggacgtgtcccacgaagagcctgacgtgaagttcaactggtacgtggatggcgtggaggtgcacaatgctaagactaaaccacgcgaagagcagtataatagtacataccgagtcgtgtcagtcctgacagtgctgcaccaggattggctgaacggcaaggagtataagtgcaaggtgtctaacaaggccctgcccgcccctatcgagaaaacaattagcaaggccaaagggcagccacgggaaccccaggtctacactctgccaccctcaagagatgaactgactaagaaccaggtcagcctgacctgtctggtgaaaggcttctaccccagcgacatcgccgtggagtgggaaagtaacggccagcctgagaataactacaagactacccctccagtgctggatagcgacgggtccttcttcctgtatagcaagctgacagtggacaaatcccgctggcagcagggaaacgtcttttcctgttctgtgatgcatgaggccctgcacaatcattacacccagaagagtctgtcactgagccccggcaaatgataagcttgcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 13 - protein, 107 aa, Artificial Sequence [sequence omitted]

SEQ ID NO: 14 - protein, 330 aa, Artificial Sequence; identical to SEQ ID NO: 11
  Synthetic Construct

SEQ ID NO: 15 - DNA, 6769 bp, Artificial Sequence
  pN3019 TBG 4q7sc1
  repeat_region (1)..(130): 5′-ITR
  promoter (205)..(913): TBG
  TATA_signal (875)..(878)
  Intron (939)..(1071): SV40 misc intron
  misc_feature (1160)..(1207): c-myc 5′UTR
  misc_feature (1219)..(1227): leader
  misc_feature (1276)..(1602): 4q7 light
  misc_feature (1597)..(1917): CL
  misc_feature (1918)..(1929): furin
  misc_feature (1927)..(2421): 4q7sc1h
  misc_feature (2419)..(2739): CH1
  misc_feature (2742)..(3408): CH2-3
  polyA_signal (3527)..(3741): Bovine Growth Hormone polyadenylation (BGH-PolyA)
  repeat_region (3829)..(3958): 3′-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccaattcgaatggccatgggacgtcgacctgaggtaattataacccgggccctatatatggatccaattcgctagcgggcactttgcactggaacttacaacacccgagcaaggacgcgactctgccgccccaccatgcgcatgcagctgctgctgctgatcgccctgagcctggccctggtgaccaacagcgatatccagatgacccagagcccagccagcctgagcgccagcgtgggagagaccgtgaccatcacctgccgggccagcgagaacatctacagctacctggcctggtaccagcagaagcagggaaagagcccacagctgctggtgtacaacgccaagaccctgatcgagggagtgccaagccggttcagcggaagcggaagcggaacccagttcagcctgaagatcaacagcctgcagccagaggatttcggaagctacttctgccagcaccacttcggaaccccattcaccttcggaagcggaaccgagctggagatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgccggaagcggcgggccccagtgaagcagaccctgaacttcgatctgctgaagctggccggagatgtggagagcaacccaggaccaatgtacagaatgcagctgctgagctgcatcgccctgagcctggccctggtgaccaacagcgaggtgcagctgcaggagagcggaccagagctggagatgccaggagccagcgtgaagatcagctgcaaggccagcggaagcagcttcaccggattcagcatgaactgggtgaagcagagcaacggaaagagcctggagtggatcggaaacatcgatacctactacggaggaaccacctacaaccagaagttcaagggaaaggccaccctgaccgtggataagagcagcagcaccgcctacatgcagctgaagagcctgaccagcgaggatagcgccgtgtactactgcgcccggagcgcctactacggaagcaccttcgcctactggggacagggaaccctggtgaccgtgagcagcgctagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtatagcctgagcagcgtggtgaccgtgccaagcagcagcctgggaacccagacctacatctgcaacgtgaaccacaagccaagcaacaccaaggtggataagaaggtggagccaaagagctgcgataagacccacacgtgccctccatgtccagcccccgaactgctgggcgggcctagcgtgttcctgtttccccctaagcctaaagatacactgatgattagtagaaccccagaggtcacatgcgtggtcgtggacgtgtcccacgaagagcctgacgtgaagttcaactggtacgtggatggcgtggaggtgcacaatgctaagactaaaccacgcgaagagcagtataatagtacataccgagtcgtgtcagtcctgacagtgctgcaccaggattggctgaacggcaaggagtataagtgcaaggtgtctaacaaggccctgcccgcccctatcgagaaaacaattagcaaggccaaagggcagccacgggaaccccaggtctacactctgccaccctcaagagatgaactgactaagaaccaggtcagcctgacctgtctggtgaaaggcttctaccccagcgacatcgccgtggagtgggaaagtaacggccagcctgagaataactacaagactacccctccagtgctggatagcgacgggtccttcttcctgtatagcaagctgacagtggacaaatcccgctggcagcagggaaacgtcttttcctgttctgtgatgcatgaggccctgcacaatcattacacccagaagagtctgtcactgagccccggcaaatgataagcttatggggattggtggcgacgactcctggagcccgtcagtatcggcggaattccagctgagcgccggtcgctaccattaccagttggtctggtgtcaaaaataaggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 16 - DNA, 6775 bp, Artificial Sequence
  TBG 2g4sc1
  repeat_region (1)..(130): 5′-ITR
  promoter (205)..(913): TBG
  TATA_signal (875)..(878)
  Intron (939)..(1071): SV40 intron
  misc_feature (1160)..(1207): c-myc 5′UTR
  misc_feature (1219)..(1227): leader
  misc_feature (1276)..(1596): 2g4scl
  misc_feature (1597)..(1917): CL
  misc_feature (1918)..(1929): furin
  misc_feature (1927)..(2427): 2g4sc1 heavy
  misc_feature (2425)..(2745): CH1
  misc_feature (2748)..(3414): CH2-3
  polyA_signal (3533)..(3747): bovine growth hormone polyA
  repeat_region (3835)..(3964): 3′-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccaattcgaatggccatgggacgtcgacctgaggtaattataacccgggccctatatatggatccaattcgctagcgggcactttgcactggaacttacaacacccgagcaaggacgcgactctgccgccccaccatgcgcatgcagctgctgctgctgatcgccctgagcctggccctggtgaccaacagcgatatccagatgacccagagcccagccagcctgagcgtgagcgtgggagagaccgtgagcatcacctgccgggccagcgagaacatctacagcagcctggcctggtaccagcagaagcagggaaagagcccacagctgctggtgtacagcgccaccatcctggccgatggagtgccaagccggttcagcggaagcggaagcggaacccagtacagcctgaagatcaacagcctgcagagcgaggatttcggaacctactactgccagcacttctggggaaccccatacaccttcggaggaggaaccaagctggagatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgccggaagcggcgggccccagtgaagcagaccctgaacttcgatctgctgaagctggccggagatgtggagagcaacccaggaccaatgtacagaatgcagctgctgagctgcatcgccctgagcctggccctggtgaccaacagccaggtggccctggaggagagcggaggaggactgatgcagccaggaggaagcatgaagctgagctgcgtggccagcggattcaccttcagcaactactggatgaactgggtgcggcagagcccagagaagggactggagtgggtggccgagatccggctgaagagcaacaactacgccacccactacgccgagagcgtgaagggacggttcaccatcagccgggatgatagcaagcggagcgtgtacctgcagatgaacaccctgcgggccgaggataccggaatctactactgcacccggggaaacggaaactaccgggccatggattactggggacagggaaccagcgtgaccgtgagcagcgctagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtatagcctgagcagcgtggtgaccgtgccaagcagcagcctgggaacccagacctacatctgcaacgtgaaccacaagccaagcaacaccaaggtggataagaaggtggagccaaagagctgcgataagacccacacgtgccctccatgtccagcccccgaactgctgggcgggcctagcgtgttcctgtttccccctaagcctaaagatacactgatgattagtagaaccccagaggtcacatgcgtggtcgtggacgtgtcccacgaagagcctgacgtgaagttcaactggtacgtggatggcgtggaggtgcacaatgctaagactaaaccacgcgaagagcagtataatagtacataccgagtcgtgtcagtcctgacagtgctgcaccaggattggctgaacggcaaggagtataagtgcaaggtgtctaacaaggccctgcccgcccctatcgagaaaacaattagcaaggccaaagggcagccacgggaaccccaggtctacactctgccaccctcaagagatgaactgactaagaaccaggtcagcctgacctgtctggtgaaaggcttctaccccagcgacatcgccgtggagtgggaaagtaacggccagcctgagaataactacaagactacccctccagtgctggatagcgacgggtccttcttcctgtatagcaagctgacagtggacaaatcccgctggcagcagggaaacgtcttttcctgttctgtgatgcatgaggccctgcacaatcattacacccagaagagtctgtcactgagccccggcaaatgataagcttatggggattggtggcgacgactcctggagcccgtcagtatcggcggaattccagctgagcgccggtcgctaccattaccagttggtctggtgtcaaaaataaggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 17 - protein, 118 aa, Mus musculus
  misc_feature (28)..(32): Xaa can be any naturally occurring amino acid
  misc_feature (47)..(65): Xaa can be any naturally occurring amino acid
  misc_feature (98)..(107): Xaa can be any naturally occurring amino acid
LEESGGGLVQPGGSMKLSCVASGFTFSXXXXXWVRQSPEKGLEWVAXXXXXXXXXXXXXXXXXXXRFTISRDDSKSSVYLQMNNLRAEDTGIYYCTRXXXXXXXXXXWGQGTSVTVSS

SEQ ID NO: 18 - protein, 118 aa, Homo sapiens
  misc_feature (28)..(32): Xaa can be any naturally occurring amino acid
  misc_feature (47)..(65): Xaa can be any naturally occurring amino acid
  misc_feature (98)..(107): Xaa can be any naturally occurring amino acid
LVESGGGLVQPGGSLRLSCAASGFTFSXXXXXWVRQAPGKGLEWVGXXXXXXXXXXXXXXXXXXXRFTISRDDSKSITYLQMNSLRAEDTAVYYCTRXXXXXXXXXXWGQGTSVTVSS

SEQ ID NO: 19 - protein, 107 aa, Mus musculus
  misc_feature (24)..(34): Xaa can be any naturally occurring amino acid
  misc_feature (50)..(56): Xaa can be any naturally occurring amino acid
  misc_feature (89)..(97): Xaa can be any naturally occurring amino acid
DIQMTQSPASLSVSVGETVTITCXXXXXXXXXXXWYQQKQGKSPQLLVYXXXXXXXGVPSRFSGSGSGTQYSLKINSLQSEDFGSYYCXXXXXXXXXFGGGTKLEIK

SEQ ID NO: 20 - protein, 107 aa, Homo sapiens
  misc_feature (24)..(34): Xaa can be any naturally occurring amino acid
  misc_feature (50)..(56): Xaa can be any naturally occurring amino acid
  misc_feature (89)..(97): Xaa can be any naturally occurring amino acid
DIQMTQSPSSLSASVGDRVTITCXXXXXXXXXXXWYQQKPGKAPKLLLYXXXXXXXGVPSRFSGSGSGTDYTLTISSLQPEDFATYYCXXXXXXXXXFGGGTKLEIK

SEQ ID NO: 21 - protein, 119 aa, Mus musculus
  misc_feature (31)..(35): Xaa can be any naturally occurring amino acid
  misc_feature (50)..(66): Xaa can be any naturally occurring amino acid
  misc_feature (99)..(108): Xaa can be any naturally occurring amino acid
EIQLQQSGPELVKPGASVKVSCKASGYSFTXXXXXWVKQSHGKSLEWIGXXXXXXXXXXXXXXXXXKATLTVDKSSSTAFMHLNSLTSEDSAVYYCARXXXXXXXXXXWGQGTLVTVSA

SEQ ID NO: 22 - protein, 118 aa, Homo sapiens
  misc_feature (30)..(34): Xaa can be any naturally occurring amino acid
  misc_feature (49)..(65): Xaa can be any naturally occurring amino acid
  misc_feature (98)..(107): Xaa can be any naturally occurring amino acid
VQLVQSGAEVKKPGASVKVSCKASGYTFTXXXXXWVRQAPGQGLEWMGXXXXXXXXXXXXXXXXXRVTMTRDTSISTAYMELSRLRSDDTAVYYCARXXXXXXXXXXWGQGTLVTVSA

SEQ ID NO: 23 - protein, 107 aa, Mus musculus
  misc_feature (24)..(34): Xaa can be any naturally occurring amino acid
  misc_feature (50)..(56): Xaa can be any naturally occurring amino acid
  misc_feature (89)..(97): Xaa can be any naturally occurring amino acid
DIQMTQSPASLSASVGETVTITCXXXXXXXXXXXWYQQKQGKSPQLLVYXXXXXXXGVPSRFSGSGSGTQFSLKINSLQPEDFGSYYCXXXXXXXXXFGSGTELEIK

SEQ ID NO: 24 - protein, 106 aa, Homo sapiens
  misc_feature (23)..(33): Xaa can be any naturally occurring amino acid
  misc_feature (49)..(55): Xaa can be any naturally occurring amino acid
  misc_feature (88)..(96): Xaa can be any naturally occurring amino acid
IQLTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGSGTELEIK

SEQ ID NO: 25 - protein, 121 aa, Artificial Sequence
  Humanized 2G4VH
QVQLVESGGGLMQPGGSLRLSCAASGFTFSNYWMNWVRQAPGKGLEWVGEIRLKSNNYATHYAESVKGRFTISRDDSKRITYLQMNTLRAEDTAVYYCTRGNGNYRAMDYWGQGTSVTVSS

SEQ ID NO: 26 - protein, 107 aa, Artificial Sequence
  Humanized 2G4VL
DIQMTQSPSSLSASVGDRVSITCRASENIYSSLAWYQQKPGKAPKLLLYSATILADGVPSRFSGSGSGTDYTLTISSLQPEDFATYYCQHFWGTPYTFGGGTKLEIK

SEQ ID NO: 27 - protein, 119 aa, Artificial Sequence
  Humanized 4G7VH
EVQLVESGAEVEMPGASVKISCKASGSTFTGFSMNWVRQANGQGLEWMGNIDTYYGGTTYNQKFKGRVTMTRDTSISTAYMQLKRLRSDDTAVYYCARSAYYGSTFAYWGQGTLVTVSS

SEQ ID NO: 28 - protein, 107 aa, Artificial Sequence
  Humanized 4G7VL
DIQLTQSPSSLSASVGDRVTITCRASENIYSYLAWYQQKPGKAPKLLIYNAKTLIEGVPSRFSGSGSGTDFTLTISSLQPEDFATYFCQHHFGTPFTFGSGTELEIK

SEQ ID NO: 29 - protein, 736 aa, Artificial Sequence
  capsid protein vp1 (adeno-associated virus 9)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL